(19) United States
(12) Patent Application Publication
(10) Pub. No.: US 2024/0125725 A1
(43) Pub. Date: Apr. 18, 2024

(54) APPARATUS, METHOD, AND SYSTEM FOR DETECTION OF THE ISOTOPIC COMPOSITION OF LIQUIDS

(71) Applicant: AquaSensing Inc., Waterloo (CA)

(72) Inventors: Xiaoye ZHAO, Waterloo (CA); Walter W. DULEY, Waterloo (CA); Yunhong ZHOU, Waterloo (CA)

(73) Assignee: AquaSensing Inc., Waterloo, ON (CA)

(21) Appl. No.: 18/379,298

(22) Filed: Oct. 12, 2023

Related U.S. Application Data
(60) Provisional application No. 63/415,603, filed on Oct. 12, 2022.

(51) Int. Cl. G01N 27/10; G01N 33/18

(52) U.S. Cl. CPC G01N 27/10; G01N 33/18; G01N 2033/0093

(57) ABSTRACT

The present disclosure is directed towards a method, system, and apparatus for distinguishing between isotopically substituted liquids, such as H₂O and D₂O, and sensing other isotopologues of water, such as H₂¹⁸O. In an embodiment, the electrical output characteristics of the apparatus or device are used as diagnostic signals to distinguish between isotopic compositions and isotopologues of a liquid. In an embodiment, water droplets with volume of several micro litre are applied to the apparatus or device generating some electrical signals which show differences between the application of H₂O and D₂O, for example. One advantage of the present method and system is that the configuration is simple and portable, with no need for complicated equipment and operation protocols.

### REFERENCE TO RELATED APPLICATIONS

This application claims the benefit of U.S. Provisional Application No. 63/415,603 filed on Oct. 12, 2022, and entitled AN APPARATUS, METHOD, AND SYSTEM FOR DETECTION OF THE ISOTOPIC COMPOSITION OF LIQUIDS, the entirety of which is incorporated herein by reference.

### FIELD

The present disclosure is generally directed towards an apparatus, method, and system for detection of the isotopic composition of liquids.

### BACKGROUND

In various industrial applications, it is useful to distinguish between different isotopes of liquids. For example, heavy water (D₂O) is used as a coolant and moderator in nuclear reactors to control the way neutrons propagate in fissionable material and to slow down neutrons produced during fission reactions. This further helps in sustaining the chain reaction allowing the nuclear reactor to operate efficiently and stably. As it is typically found wherever a nuclear chain reaction occurs, sensing D₂O or distinguishing D₂O from H₂O can be significant for building a fission reactor and avoiding the conditions that pre-exist prior to a nuclear accident.

For a heavy water (D₂O) molecule, hydrogen atoms in regular water are replaced by deuterium atoms, but these two isotopologues of water have the same visual appearance and have similar chemical reactivity. As a result, the presence of D₂O can only be detected through costly tests like atomic absorption spectroscopy, mass spectrometry, nuclear magnetic resonance spectroscopy, and infrared spectroscopy. These conventional methods suffer from several limitations including the necessity of transferring samples to a fixed site, the use of expensive specialized analytical instruments, and complex analytical protocols requiring trained technicians.

Therefore, there is a need for an inexpensive stand-alone detector which can provide real-time analysis that can indicate the presence of certain isotopes of a liquid, such as D₂O as distinguished from H₂O, at the point of generation, which may include physically inaccessible locations.

### SUMMARY

This disclosure is directed towards a method and system for distinguishing between isotopically substituted liquids, such as H₂O and D₂O, and sensing other isotopologues of water, such as H₂¹⁸O.

In one embodiment, the electrical output characteristics of the apparatus or device are used as diagnostic signals to distinguish between isotopes and isotopologues of water. Water droplets with volume of several micro litres are applied to the device generating characteristic electrical signals which show differences between the application of H₂O and D₂O to a sensor, for example. One advantage of the present method and system is that the configuration is simple and portable for use in the field, with no need for complicated equipment and operation protocols.

In an embodiment, an apparatus or device for practicing the method contains a single porous middle layer placed in close contact with upper and lower inert or substantially inert electrodes. In one embodiment, water (e.g. H₂O, D₂O or a mixture of H₂O and D₂O) droplets are applied to the porous middle layer at a location adjacent to the inert upper electrode. The flow of water into the porous middle layer results in the generation of a voltage between the two inert electrodes. The amplitude and time-dependence of this voltage can be measured using a standard device such as a digital multimeter, an oscilloscope or simply a data acquisition card connected to the upper and lower electrodes.

In another embodiment, the apparatus or device produces two sequential voltage signals when a water droplet is applied at a location adjacent to the top electrode. The first voltage pulse (Vₛₕₐᵣₚ) is sharp with a time duration of hundreds of microseconds, and the second voltage pulse (V_{broad} or V_{wide}) is much wider by comparison, with a time duration of hundreds of milliseconds. The amplitude of V_{wide}, the ratio of the amplitude of Vₛₕₐᵣₚ to the amplitude of V_{wide}, the time dependence of Vₛₕₐᵣₚ, and shape of the V_{wide} pulse are each found to be different for D₂O and H₂O droplets.

In another embodiment, the middle layer is porous and hydrophilic and consists of a structure assembled from nano- and/or micro particles and/or wires. In a further aspect, nano- and/or micro-channels are fabricated into the middle layer allowing water to rapidly flow from the top side to the bottom side of the middle layer. In a further aspect, the middle nanostructure is fabricated with a compression method, whereby the nano- and/or micro-materials are compressed into a disk using a heavy block material, a hand press or a controllable machine press. In one embodiment, a universal press or stamping machine is used to compress the nano- and/or micro-material into a disc, with compression pressures ranged from about 3 MPa to 9 MPa. In this illustrative embodiment, an optimized compression pressure of 6 MPa is used for fabricating the porous middle layer.

In another embodiment, the thickness of the porous middle layer can be controlled by the mass of the nanoparticles in the precursor porous sample before compression. Variation of this mass permits the thickness of the porous middle layer to range from about 100 micrometers to several millimeters after compression. In another aspect, a specific mass of the nano- and/or micro-sized material is put into a mould for compressing, fabricating porous layer with thickness ranged from about 0.4 mm to 1.8 mm In one embodiment, the thickness is selected as 0.4 mm by considering both the mechanical properties of the middle layer and the sensing efficiency of the detector on application of water droplets having a standard volume of 8 μL, for example. The operating conditions of the apparatus or device are determined by a various factors including porous layer thickness, time interval between water droplets and a selected standard volume of each droplet, and this sensor can work with different combinations of above parameters. In another aspect, the volume of water droplets can be several or tens of microliters.

In another embodiment, the material that makes up the middle layer includes Al₂O₃ nanoparticles with diameters of hundreds of nanometers, Al₂O₃ nanowires, TiO₂ nanoparticles/nanowires, ZnO nanoparticles/nanowires, or SiO₂ nanoparticles. In one embodiment, the middle layer is fabricated from Al₂O₃ nanoparticles with a diameter of 200-300 nm, and this layer is porous with abundant of nanochannels and is super hydrophilic facilitating unhindered water flow from the top surface to the bottom surface of the middle layer.

In another embodiment, the electrode material includes carbon paper, carbon cloth, gold or gold-coated silicon wafer, platinum or platinum-coated silicon wafer, silver, copper, or stainless steel. In a further aspect, the bottom electrode is a flaky material with a surface area the same or larger than that of the nanoparticle layer. In another aspect, the top electrode is porous and hydrophilic for water penetration.

In another embodiment, the top electrode geometry is configured to permit a rod-shaped or point contact with the middle layer enabling the application of liquid directly onto the top surface of the middle layer. In another aspect, the top electrode may be slightly separated from the surface of the middle layer such that the gap between the electrode and the middle layer constitutes a capacitor.

In another embodiment, the voltage output between the top and bottom electrodes originates from the interactions between water molecules in the applied liquid and the top electrode as the water fills the gap between the top electrode and the porous middle layer. In a further aspect, Vₛₕₐᵣₚ originates from charges collected by the top electrode where these charges are produced during a liquid pipetting process, while V_{wide} originates from the flow of water through the porous middle layer.

### BRIEF DESCRIPTION OF THE DRAWINGS

The present invention, and the objects of the invention will become apparent when consideration is given to the following detailed description thereof. Such description makes reference to the annexed drawings, wherein:

FIG. 1A shows an illustration of one structure of the heavy water sensor, porous and flaky top electrode. The arrows schematically indicate the routes used as water passes through the porous medium.

FIG. 1B shows an illustration of one structure of the heavy water sensor, a rod top electrode is placed to form a point contact with the porous middle layer. The arrows schematically indicate the routes used as water passes through the porous medium.

FIG. 1C shows SEM of Al₂O₃ film: (a) and (b) surface. (c) and (d) cross-section. (e) EDS of the point marked in (b).

FIG. 2A shows a flowchart of the process for making a sensor for heavy water detection via direct compression.

FIG. 2B shows a structure of the mould used in the direct compression process.

FIG. 3 shows a flowchart of the process for making a sensor for heavy water detection via vacuum filtration where carbon paper acts as the filter and lower electrode.

FIG. 4 shows a flowchart of the process for making a sensor for heavy water detection using a vacuum filtration method where filter paper is used to collect the layer of nanoparticles.

FIG. 5 shows an open circuit voltage (OCV) measured by a multimeter when sensing (a) H₂O and (b) D₂O. The equilibrium state is reached after application of five droplets and is indicated by the dashed lines. (c) Comparison of the OCV generated in the equilibrium state from different sensors having the same nominal composition and structure on application of H₂O and D₂O droplets. The detector configuration is as shown in FIG. 1B.

FIG. 6 shows characteristics of the OCV pulses generated from application of 8 μL volume droplets of (a) H₂O and (b) D₂O, measured by an oscilloscope (200 KHz bandwidth). (c) Calculated ratio of the peak amplitudes of Vₛₕₐᵣₚ to V_{wide}

FIG. 7A shows time dependence of Vₛₕₐᵣₚ for several devices when applying 8 μL volume droplets of H₂O and D₂O droplets to the detector as shown in FIG. 1B.

FIG. 7B shows fitting results of the Vₛₕₐᵣₚ. (a) and (b) Fits to the experimental data using eqns. 1 and 2. (c) Plot of the maximum amplitude of Vₛₕₐᵣₚ vs. rise time tc. (d) Plot of Vg vs. τg

FIG. 7C shows output of dVₛₕₐᵣₚ/dt. (a) H₂O. (b) D₂O.

FIG. 8 shows the time dependence of V_{wide} with a time window of 0-0.15 s and the corresponding Fast Fourier Transform (FFT) output showing changes in the frequency domain: (a) and (b) H₂O, (c) and (d) D₂O. The labels 1-4 correspond to the sequential application of 8 μL droplets. The dashed boxes indicate regions of the frequency spectrum that can be used to identify D₂O.

FIG. 9A shows the relationship between middle layer thickness and the mass of the Al₂O₃ nanoparticles before compression.

FIG. 9B shows characteristics of OCV pulses generated from application of 8 μL volume droplets of H₂O and D₂O measured by detectors fabricated with different thicknesses of the Al₂O₃ layer: (a) and (e) 0.4 mm. (b) and (f) 0.6 mm. (c) and (g) 1.3 mm. (d) and (k) 1.8 mm.

FIG. 10A shows characteristics of OCV pulses generated from application of 8 μL volume droplets of H₂O and D₂O measured from detectors fabricated with Ag top electrode and different bottom electrodes: (a) and (b) Cu. (c) and (d) ITO-coated PET. (e) and (f) Pt.

FIG. 10B shows characteristics of OCV pulses generated from application of 8 μL volume droplets of H₂O and D₂O measured from detectors fabricated with Cu top electrode and carbon paper bottom electrode.

FIG. 11A shows an OCV generated with application of 8 μL droplets of H₂O when the detector is operated at 50° C. The temperature of each water droplet is 25° C.

FIG. 11B shows an OCV generated with application of 8 μL droplets of D₂O when the detector is operated at 50° C. The temperature of each water droplet is 25° C.

FIG. 11C shows a temporal evolution of the voltage pulses generated from application of 8 droplets of (a) H₂O and (b) D₂O on the detector as shown in FIG. 1B operating at a 50° C. The time interval between applied water droplets was 2.5 min. The droplet temperature was 25° C.

FIG. 12 shows a time sequence of the leading edge of the V_{wide} response on exposure to a series of 8 μL droplets of H₂O and H₂¹⁸O applied to the detector as shown in FIG. 1A. The arrows indicate the sequence of droplet application.

FIG. 13 shows characteristics of the OCV pulses generated from application of 8 μL volume droplets of (a) H₂O and (b) D₂O onto an Ag-Al₂O₃ nanowire-carbon paper device.

FIG. 14 shows concentration dependence of the OCV generated on application of droplets of D₂O—H₂O solutions. (a) D₂O concentration from 0.0 vol. % to 95 vol. %. (b) Expanded scale at low D₂O concentration. (c) Average voltage calculated from the first seven OCV pulses, second seven OCV pulses and the OCV generated from application of deionized (DI) water after the exposure of the mixed droplets.

FIG. 15 shows the OCV response on application of 8 μL droplets containing a mixture of H₂O and D₂O. (a) 100 ppm of D₂O and (b) 50 ppm of D₂O.

FIG. 16 shows characteristics of the OCV pulses generated from application of 8 μL droplets of the D₂O—H₂O mixture containing 100 ppm of D₂O. (a) and (b) OCV response for the first droplet to the fourteenth droplet. (c) Summary of peak OCV and calculated ratio of the peak amplitudes of Vₛₕₐᵣₚ to V_{wide}. The OCV responses were measured by an oscilloscope (200 KHz bandwidth), and the middle layer thickness is 1.3 mm.

FIG. 17A shows (a) O1s and (b) A12p core spectra of the original porous Al₂O₃ film and an Al₂O₃ film after exposure to H₂O and D₂O.

FIG. 17B shows s schematic showing the differences in the quantity of surface function groups, surface charge density and characteristics of the surface potential.

FIG. 18 shows (a) a schematic for the generation of Vₛₕₐᵣₚ. (b) Relationship between the maximum amplitude of Vₛₕₐᵣₚ and rise time of Vₛₕₐᵣₚ on application of the H₂O and D₂O droplets to the metal-metal system without a nanoparticle film. (e) Relationship between the maximum amplitude of Vₛₕₐᵣₚ and rise time of Vₛₕₐᵣₚ when on application of the H₂O and D₂O droplets to the Ag and Cu electrode without a nanoparticle film and bottom electrode.

FIG. 19A shows a system developed for the sensor.

FIG. 19B shows a display of voltage signals on application of H₂O and D₂O droplets measured with a sampling rate of 200 Hz.

FIG. 19C shows the collected voltage signals at a sampling rate of 500 kHz. Changes in dVₛₕₐᵣₚ/dt enable a distinction between H₂O and D₂O.

FIG. 20 shows a schematic block diagram of a generic computer which may provide a platform for one or more embodiments.

### DETAILED DESCRIPTION

As noted above, the present disclosure is directed towards a method and system for distinguishing between liquids having different isotopic composition, such as H₂O and D₂O, and sensing other isotopologues of water, such as H₂¹⁸O.

In one embodiment, the electrical output characteristics of the apparatus or device are used as diagnostic signals to distinguish between isotopes and isotopologues of water. Water droplets with volume of several micro litre are applied to the apparatus or device generating some electrical signals which show differences between the application of H₂O and D₂O, for example. One advantage of the present method and system is that the configuration is simple and portable, with no need for complicated equipment and operation protocols.

In an embodiment, an apparatus or device for practicing the method contains a single porous middle layer placed in close contact with upper and lower inert electrodes. In one embodiment, water (e.g. H₂O, D₂O or a mixture of H₂O and D₂O) droplets are applied to the porous middle layer at a location adjacent to the inert upper electrode. The flow of water into the porous middle layer results in the generation of a voltage between the two inert electrodes. The amplitude and time-dependence of this voltage can be measured using a standard device such as a digital multimeter, an oscilloscope or simply a data acquisition card connected to the upper and lower electrodes.

In another embodiment, the apparatus or device produces two sequential voltage signals when a water droplet is applied at a location adjacent to the top electrode. The first voltage pulse (Vₛₕₐᵣₚ) is sharp with a time duration of hundreds of microseconds, and the second voltage pulse (V_{wide}) is much wider with a time duration of hundreds of milliseconds. The amplitude of V_{wide}, the ratio of the amplitude of Vₛₕₐᵣₚ to the amplitude of V_{wide}, the time dependence of Vₛₕₐᵣₚ, and shape of the V_{wide} pulse are each found to be different for D₂O and H₂O droplets.

In another embodiment, the middle layer is porous and hydrophilic and consists of a structure assembled from nano- and/or micro particles and/or wires. In a further aspect, nano- and/or micro-channels are fabricated into the middle layer allowing water to rapidly flow from the top side to the bottom side of the middle layer. In a further aspect, the middle nanostructure is fabricated with a compression method, whereby the nano- and/or micro-materials are compressed into a disk using a heavy block material, a hand press or a controllable machine press. In one embodiment, a universal press or stamping machine is used to compress the nano- and/or micro-material into a disc, with compression pressures ranging from 3 MPa to 9 MPa. In this illustrative embodiment, an optimized compression pressure of 6 MPa is used for fabricating the porous middle layer. However, it will be understood that other pressures may be used for other embodiments.

In another embodiment, the thickness of the porous middle layer can be controlled by the mass of the nanoparticles in the precursor porous sample before compression. Variation of this mass permits the thickness of the porous middle layer to range from about 100 micrometers to several millimeters after compression. In another aspect, a specific mass of the nano- and/or micro-sized material is put into a mould for compressing. In one embodiment, the thickness is optimized as 0.4 mm by considering both the mechanical properties of the middle layer and the sensing efficiency of the detector on application of water droplets having a selected standard volume of 8 μL at a time interval of 10 min. In another aspect, the volume of water droplets can be several or tens of microliter.

In another embodiment, the material that makes up the middle layer includes Al₂O₃ nanoparticles, Al₂O₃ nanowires, TiO₂ nanoparticles/nanowires, ZnO nanoparticles/nanowires, or SiO₂ nanoparticles. In one embodiment, the middle layer is fabricated from Al₂O₃ nanoparticles with a diameter of 200 nm, as the nanoporous layer produced by this size is mechanically stable and can produce repeatable high voltage outputs, and this layer is porous with abundant of nanochannels and is super hydrophilic facilitating unhindered water flow from the top surface to the bottom surface of the middle layer.

In another embodiment, the electrode material includes carbon paper, carbon cloth, gold or gold-coated silicon wafer, platinum or platinum-coated silicon wafer, silver, copper, or stainless steel. In a further aspect, the bottom electrode is a flaky material with a surface area the same or larger than that of the nanoparticle layer. In another aspect, the top electrode is porous and hydrophilic for water penetration.

In another embodiment, the top electrode geometry is configured to permit a rod-shaped or point contact with the middle layer enabling the application of liquid directly onto the top surface of the middle layer. In another aspect, the top electrode may be slightly separated from the surface of the middle layer such that the gap between the electrode and the middle layer constitutes a capacitor.

In another embodiment, the voltage output between the top and bottom electrodes originates from the interactions between water molecules in the applied liquid and the top electrode as the water fills the gap between the top electrode and the porous middle layer. In a further aspect, Vₛₕₐᵣₚ originates from charges collecting by the top electrode and these charges are produced during a liquid pipetting process, while V_{wide} originates from the flow of water through the porous middle layer.

In the following description, various illustrative embodiments will show how the method and system may be practiced. It is to be understood that other embodiments may be utilized, and structural changes may be made without departing from the scope of the invention.

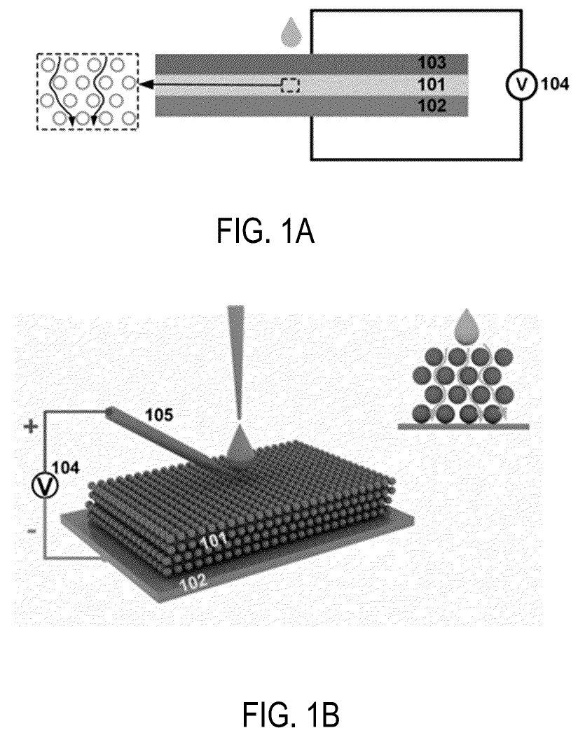

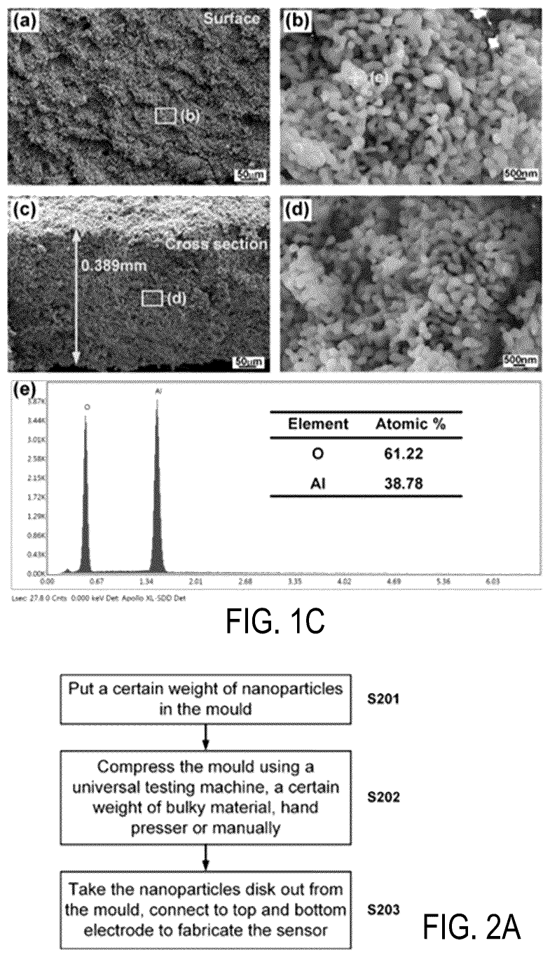

As exemplary embodiments, FIG. 1A and FIG. 1B show the schematic view of the sensing component, whereby the difference between these two configurations is the type of the top electrode. The sensor presents a sandwich structure which includes a middle nanoparticles layer (101), a bottom flaky electrode (102), and a top electrode (103 and 105). The top electrode can either be a porous flaky and hydrophilic electrode (103) that can allow liquid to flow through or be a rod electrode (105) that makes a point contact with the middle layer with which the tested liquid can be applied directly to the middle layer. Two simple methods are adopted to form a porous middle layer using nano- and/or micro-scale materials: direct compression and vacuum filtration. Control over the thickness and porous morphology of the middle layer can be achieved in these two ways. The term “porous morphology” in the above and elsewhere refers to a structure which contains nanoscale or microscale channels between single nanomaterial units, as shown in FIG. 1A, FIG. 1B and SEM microstructures shown in FIG. 1C. Other film formation methods which can form this kind of structure can also be adopted.

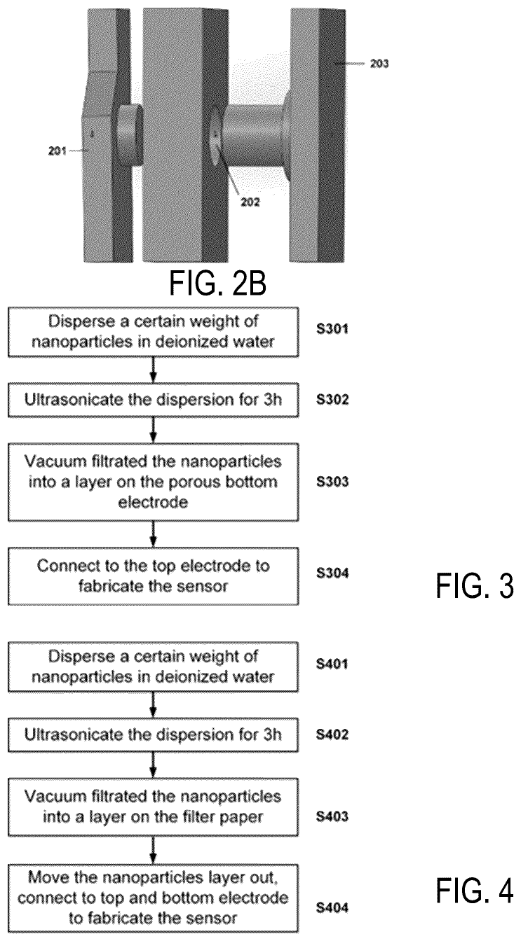

In FIG. 2A, the porous middle layer is fabricated by a direct compression method, as shown in steps S201, S202, and S203. A mould is used during this process, as shown in FIG. 2B. The mould is composed of three pieces, 201 is the base stationary plate, 202 is the middle plate that containing the cavity and 203 is the top movable plate. A certain weight of nanomaterial is put into the mould cavity, the mould is shaken a few times to uniformly distribute the nanoparticles in the cavity. After putting on the top movable plate, the mould is compressed using a universal tensile machine, a certain weight of bulk material, hand press or the mould can be pressed manually. Then, a stacked layer of nanoparticles is formed with an abundance of interstitial nano and/or micro-scale channels. The porous middle layer is removed from the mould and connected to bottom and top electrodes to form the sensing component. The thickness of the middle layer is mainly controlled by the mass of nanoparticles, and compression pressure can also determine the thickness. The porosity of the middle layer is controlled by the compression pressure.

In FIG. 3 and FIG. 4, the porous middle layer is fabricated by vacuum filtration, whereby the main difference is the type of the filter. A certain weight of nanoparticles is dispersed in deionized water by ultrasonic vibration to separate the materials into small units and increase the porosity, uniformity and surface to volume ratio after deposition. A nano and/or micro-scale network of nanoparticles is formed by vacuum filtration such that the nanoparticles are filtered out and become stacked on the bottom carbon paper electrode forming a structure containing a high density of nano and/or micro-scale channels. The layer thickness and porosity may be controlled by adjusting the dispersion concentration, vacuum pressure, as well as the structures of single material units (particles, wires, flakes). In the process of steps S301, S302, S303, S304 shown in FIG. 3, the carbon paper acts as the filter so that the nanoparticles are directly deposited onto the bottom electrode. In another process of steps S401, S402, S403, S404 shown in FIG. 4, the adopted bottom electrodes may be nonporous, and the filter paper is selected to hold the nanoparticles. After drying in air, the nanoparticle layer is moved onto the bottom electrode to form the sensing component.

In one exemplary embodiment, 30 mg of Al₂O₃ nanoparticles each with a diameter of ˜200 nm is compressed into a disk with 10 mm in diameter and 0.4 mm in thickness. The sensing component is completed by connecting this disk to a bottom carbon paper electrode and a top Ag or other type of metal electrode contacting the top of the Al₂O₃ middle layer. The structure of the apparatus or device is shown in FIG. 1B. 101 represents the compressed middle layer. 102 represents the carbon paper that serves as the bottom electrode. This electrode can also be replaced by another relatively or substantially inert material such as gold-coated sheet, platinum-coated sheet, Ag sheet, copper sheet, carbon cloth or stainless steel. 105 represents the Ag or other conducting rod that serves as the top electrode. 104 is a multimeter or oscilloscope used for measuring the output electrical signals.

As an exemplary experimental result, the open circuit voltage (OCV) of a heavy water sensor with a structure consisting of carbon paper—0.4 mm thick Al₂O₃ nanoparticle layer-silver contact (FIG. 1B) in response to droplets were tested using the experiment setup shown in FIG. 1B. The amount of liquid applied per droplet was 8 μL and the time interval between consecutive water droplets was 10 min.

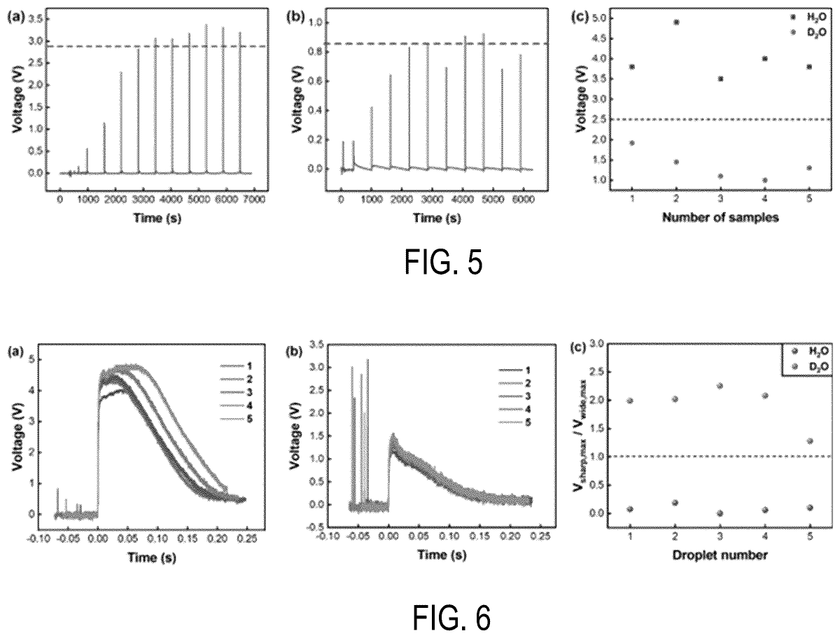

As shown in FIG. 5, the OCV generated gradually increases with the number of droplets applied consecutively to the same location on the top of the middle layer near the silver electrode. The moisture inside the Al₂O₃ middle layer reaches an equilibrium state after application of several droplets and the OCV reaches a stable value. After establishment of the equilibrium state, the maximum OCV for the detector using H₂O droplets can be high as 3.5 V while the maximum OCV for the detector using D₂O droplets is less than 1.0 V. This difference in the amplitude of the OCV for application of the same quantity of liquid D₂O and H₂O enables the distinction between D₂O and H₂O. This diagnosis is effective when the sampling time of the measuring equipment is in the range from several milliseconds to tens of milliseconds. The OCV data shown in FIG. 5 were measured using a digital multimeter with a sampling rate of 25 Hz. In the embodiment presented in FIG. 5, the internal resistance of the multimeter is 10 MΩ. From the OCV generated from multiple devices, it is concluded that the voltage generated in the equilibrium state ranges from 3 V to 5 V, and from 0.7 V to 2 V for application of H₂O droplets and D₂O droplets, respectively.

Data recording with a high sampling rate can show more detailed characteristics of the voltage signals and provide two additional diagnostics for distinguishing D₂O from H₂O. One series of OCV pulses measured using an oscilloscope is shown in FIG. 6. The sampling rate for these measurements was 200 KHz. It can be seen that two voltage pulses are generated from the application of one water droplet. The first pulse is sharp (Vₛₕₐᵣₚ) and the second pulse is wide (V_{wide}) having a time duration of ˜150 ms. The Vₛₕₐᵣₚ signal is generated when the liquid droplet first contacts the silver electrode and the surface of the Al₂O₃ layer. The V_{wide} signal is produced from a ‘streaming current’ mechanism as the water flows through the Al₂O₃ middle layer. The rise time of V_{wide}, which is determined by the spread of water in the porous medium is as short as ˜20 ms. The amplitude ratio Vₛₕₐᵣₚ/V_{wide} for each droplet in a series of consecutive droplet applications has been calculated and is shown in FIG. 6(c). It is seen that Vₛₕₐᵣₚ/V_{wide} from the application of H₂O droplets is generally smaller than 1 and that from the application of D₂O droplets is generally larger than 1. This ratio is regarded as another reliable diagnostic parameter to distinguish between H₂O and D₂O.

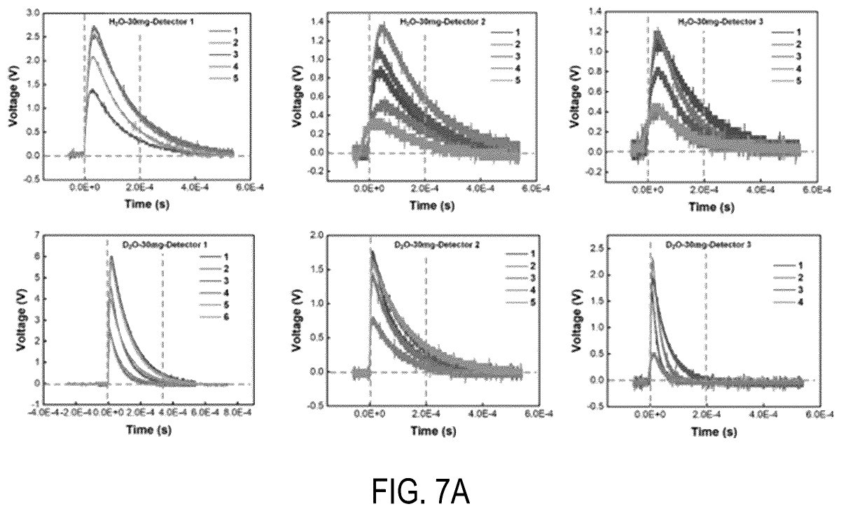

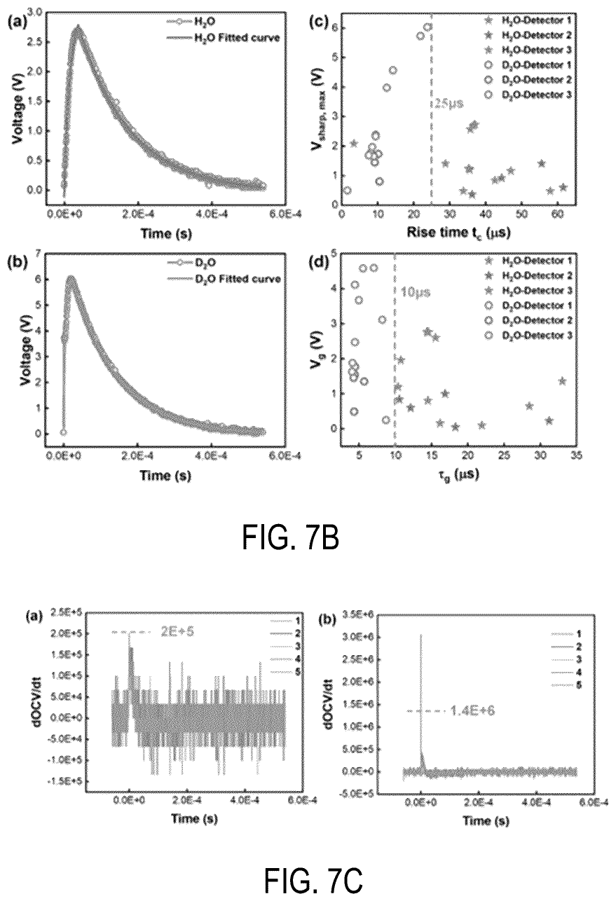

Further details on the time evolution of Vₛₕₐᵣₚ can be obtained with a sampling rate of 1 GHz. Data obtained at this sampling rate for three detectors fabricated with the same composition and electrode configuration is shown in FIG. 7A. The time dependence of V sharp, is as follows:

Vₛₕₐᵣₚ(t)=V₀+V_{d}+V_{g}(e^{-t}^{_{c}}^{/τ}^{_{g}}−e^{-t/τ}^{_{g}}),t≤t_{c} (1)

Vₛₕₐᵣₚ(t)+V₀+V_{d}e^{-(t-t}^{_{c}}^{)/τ}^{_{d}},t≤t_{c} (2)

where V₀ is the offset, t_{c} is the time required to reach the peak amplitude of Vₛₕₐᵣₚ, τg and τd are growth and decay time constants, V_{g} and V_{d} are obtained from the growth and decay amplitudes, respectively, after the fit. A plot of the maximum amplitude of Vₛₕₐᵣₚ vs. the rise time t_{c} and V_{g} vs. τ_{g} is shown in FIG. 7B following the sequential application of D₂O and H₂O droplets. These plots indicate that the time dependent characteristics of the rise of Vₛₕₐᵣₚ can be taken as another diagnostic method to distinguish between D₂O and H₂O. The derivative, dVₛₕₐᵣₚ/dt, has also been calculated (FIG. 7C), and shows that there is a significant difference in the onset of Vₛₕₐᵣₚ generated from application of H₂O droplets and D₂O droplets. This slope can also be taken as a further parameter to distinguish between H₂O and D₂O. The decay function shows a decay time constant of 19.5-134.6 μs for H₂O and 77.8-191.8 μs for D₂O droplets when Ag is used as a contact to the Al₂O₃ middle layer. Similar characteristics are obtained when the Ag electrode is replaced with an electrode having different metallic composition.

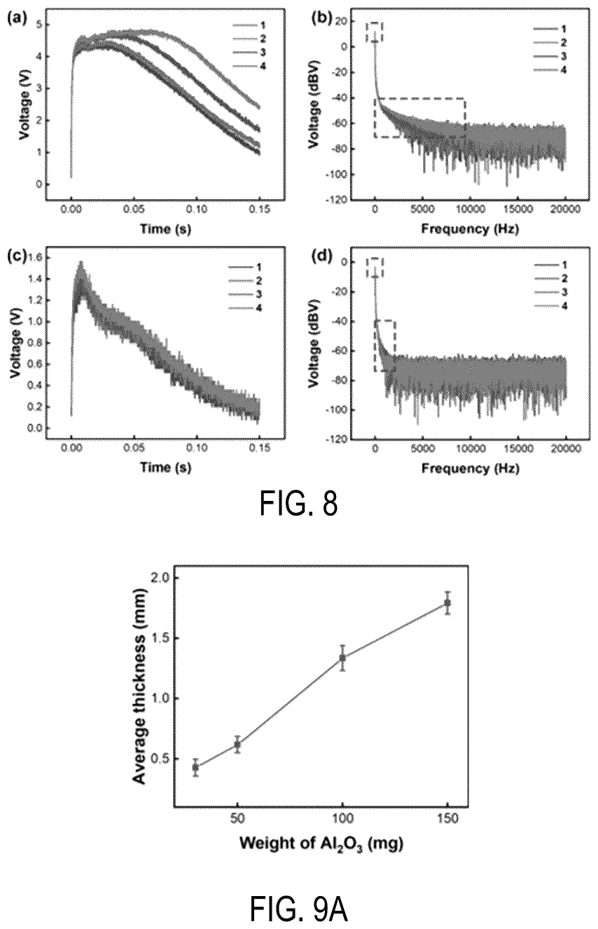

Fast Fourier transform (FFT) is an algorithm that is widely used for signal processing. The FFT converts a signal from the time domain to a representation in frequency space. In the present context, the application of FFT to the time evolution of the V_{wide} pulse shows the frequency components that can be associated with this signal (FIG. 8). These frequency components can be associated with the dynamics of the physical and chemical processes occurring in the detector that produce the OCV. For both the V_{wide} generated from the application of H₂O droplets and D₂O droplets, the frequency spectrum is characterized by a direct current (DC) component and a range of alternating frequency components extending to at least 20 kHz. The amplitude of the DC components originating from the application of H₂O droplets is ˜10 dBV, which is much higher than that from the application of D₂O droplets (˜5 dBV). This indicates that the maximum OCV amplitude from application of H₂O droplets is much higher than that from application of D₂O droplets. The amplitude of alternating current (AC) components from H₂O-triggered voltage gradually decreases as the frequency increases from ˜6 Hz to ˜10 kHz, and then stabilizes at ˜−70 dBV for the AC components at higher frequency. This transition stage from the DC component to the AC components with a stabilized voltage is faster for the D₂O-induced voltage, as shown by the dashed rectangle. The amplitude stabilized at ˜−70 dBV when the AC components come to a frequency of ˜2500 Hz or higher. The difference in the transition stage from DC component to AC ones shows that the low-frequency AC component accounts for a larger proportion in the constitution of H₂O-induced voltage signal, which makes V_{wide} from H₂O droplets is more like a step function while that from D₂O is sharp. The differences in the FFT results are regarded as an auxiliary diagnostic basis for detection of the presence of D₂O.

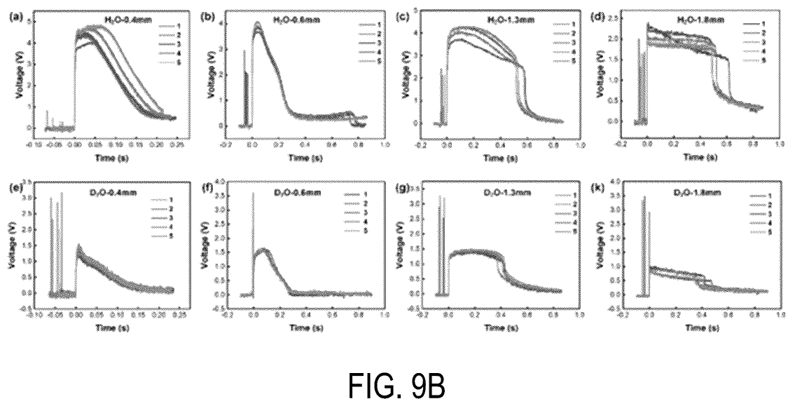

As another exemplary experimental result, the thickness of the middle layer was varied by adopting a different mass of Al₂O₃ nanoparticles during the compression process. The thickness increases with an increase in the mass of the nanoparticles, as shown in FIG. 9A. The effect of thickness on the OCV response of detector using the experimental configuration shown in FIG. 1B is summarized in FIG. 9B. This shows that the ratio of the amplitude of Vₛₕₐᵣₚ to V_{wide} is <1 for H₂O-induced voltage and >1 for D₂O-induced voltage for all the devices with different middle layer thicknesses. The amplitude of V_{wide} from H₂O droplets is also higher than that from D₂O droplets for all the devices. This shows that the amplitude of V_{wide} and the Vₛₕₐᵣₚ/V_{wide} ratio are strongly diagnostic for the presence of D₂O.

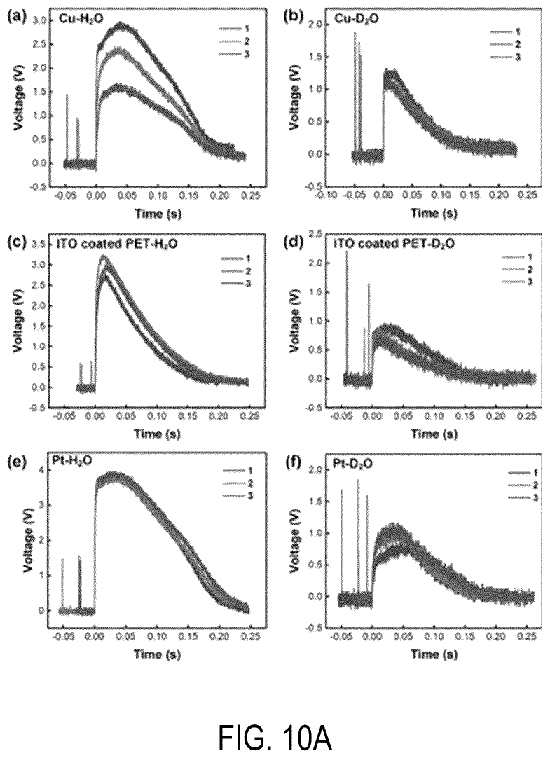

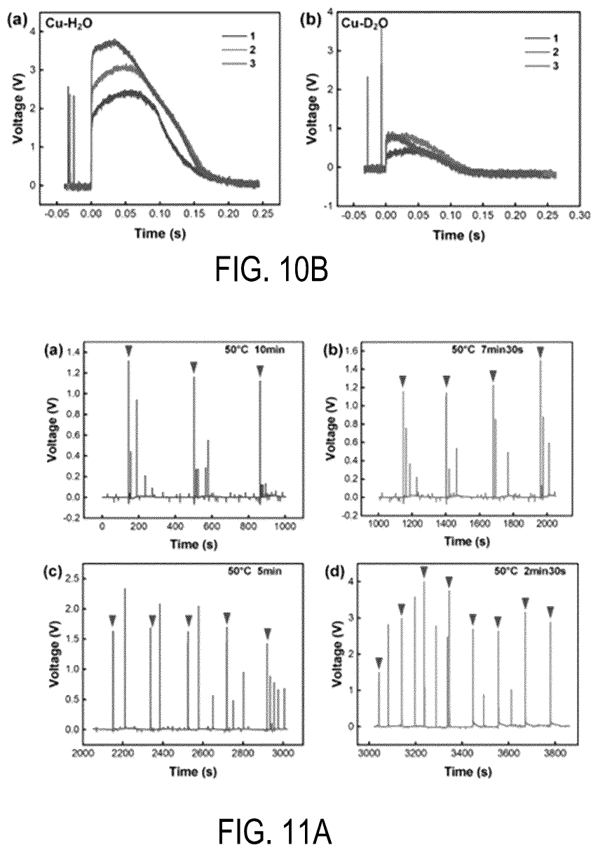

As another exemplary experimental results, different bottom electrode material (FIG. 10A) and top electrode material (FIG. 10B) in the configuration in FIG. 1B are changed, and the electrical output generated from configurations of Ag—Al₂O₃—Cu, Ag—Al₂O₃-ITO coated PET, Ag—Al₂O₃layer-Pt and Cu—Al₂O₃-carbon paper was measured. The thickness of middle layer in each of these configurations was 0.4 mm. It shows that there are differences in the amplitude of Vₛₕₐᵣₚ and V_{wide}, and the shape of V_{wide} when using different electrode materials, but the amplitude of V_{wide} from H₂O droplets is higher than that from D₂O droplets for all the configurations. The ratio of the amplitude of Vₛₕₐᵣₚ to V_{wide} is generally <1 for H₂O-induced voltage and >1 for D₂O-induced voltage for all the configurations. This shows that the amplitude of V_{wide} and the Vₛₕₐᵣₚ/V_{wide} ratio are strongly diagnostic for the presence of D₂O when use different pairs of inert electrode materials.

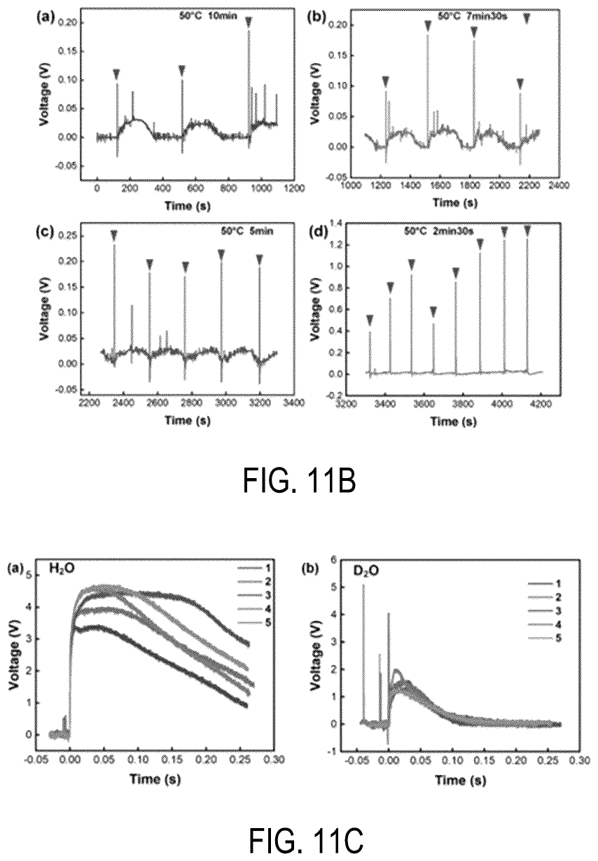

As another exemplary experimental result, the measurement using the experimental configuration in FIG. 1B is conducted at 50° C. rather than at room temperature. The measurement is conducted on a hot plate with a controlled temperature of 50° C., and the results from a silver—Al₂O₃ nanoparticles layer—carbon paper device are shown in FIG. 11A and FIG. 11B. The red triangles represent the times when water droplets were applied to the apparatus or device. The optimum time interval of applying water in this sensor system is 10-15 min, and a shorter time interval may introduce an excessive water concentration in the Al₂O₃ layer which may lead to degradation of the apparatus or device. At higher temperature, the moisture in the Al₂O₃ middle layer dries out more easily between droplet applications facilitating a shorter time interval between droplets. As shown in FIG. 11A and FIG. 11B, with a time interval of 10 min, the amplitude of V_{wide} is lower than seen in room temperature experiments, reaching only 1.3 V and 0.2 V for application of H₂O and D₂O droplets, respectively. The peak voltage amplitude remains low when the time interval is decreased to 7.5 min, and increases somewhat when the time interval is 5 min. V_{wide} increases to 4.0 V and 1.2 V when H₂O and D₂O droplets are applied every 2.5 min, and these OCV corresponds to the values generated at room temperature with a time interval of 10 min. The OCV recorded via oscilloscope with a sampling rate of 200 KHz is shown in FIG. 11C. It is apparent that the two strong indicators of D₂O, namely the amplitude of V_{wide} and Vₛₕₐᵣₚ/V_{wide} ratio are still valid diagnostic parameters at higher temperature. The on-hot plate measurements indicate that the sensor works at a high temperature with a shortened time interval between application of water droplets. The additional OCV pulses generated between water droplets, as shown in FIG. 11A and FIG. 11B, arise from the sudden evaporation of bubbles of water vapor trapped inside the porous medium.

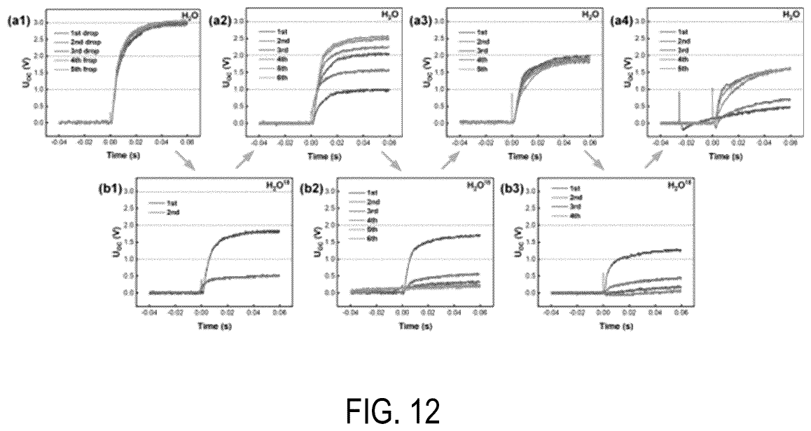

As another exemplary use for this detector, the OCV was measured on applying 8 μL of the water isotopologue H₂¹⁸O to the apparatus or device. The experimental set-up in FIG. 1A was used with an Al₂O₃ layer thickness of 1.3 mm. The time interval between droplets was 10 min. As the amplitude of Vₛₕₐᵣₚ is small using carbon paper as the top electrode for droplets of H₂O, D₂O or H₂¹⁸O, the time dependence of OCV at the leading edge of V_{wide} was used as a diagnostic parameter and the results of these measurements are shown in FIG. 12. It was found that the VOC was ˜3.0 V for the first application of a consecutive series of H₂O droplets, but OCV decreases to ≤0.5 V as H₂¹⁸O is introduced into the system. The difference in the amplitude of OCV generated by H₂O and H₂¹⁸O makes it possible to distinguish these two isotopologues of water. When the system was then exposed to H₂O again, the OCV decreased to 2.5 V which was 0.5 V lower than the original value. The VOC decreased to 2.0 V and then 1.5 V when additional H₂¹⁸O was introduced into the system.

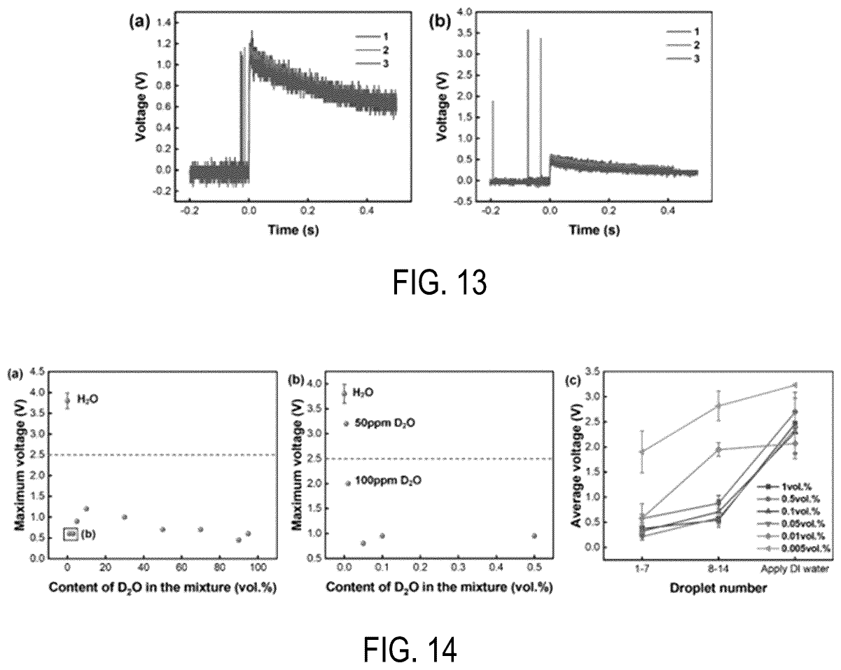

As another exemplary experimental result, the OCV generated from an Ag-Al₂O₃ nanowire-carbon paper configuration was measured on applying 8 μL H₂O and D₂O droplets onto the apparatus or device (FIG. 13). Similar to the apparatus or device containing the Al₂O₃ nanoparticle layer, V_{wide} from application of H₂O droplets has a higher amplitude than V_{wide} generated from the application of D₂O droplets. The ratio of the amplitude of Vₛₕₐᵣₚ to the amplitude of V_{wide} is ˜1 for the H₂O-induced voltage response and >1 for the D₂O-induced voltage response. This confirms that the difference in the Vₛₕₐᵣₚ/V_{wide} ratio using the apparatus or device shown in FIG. 1B is an effective way to distinguish between H₂O and D₂O.

As another exemplary experimental result, mixed solutions containing different concentrations of D₂O in H₂O liquid were prepared. The voltage response on the application of droplets of this mixture on the Ag—Al₂O₃ (1.3 mm thick layer)-carbon paper device was measured to investigate the sensitivity of this sensor to low concentrations of D₂O in the presence of H₂O. FIG. 14 shows the maximum and average OCV generated from application of droplets of D₂O—H₂O mixtures to the device. The maximum voltage is <2.5 V when the mixture contains more than 0.01 vol. % (100 ppm) of D₂O, indicating that even a small concentration of D₂O in H₂O significantly affects the detector response. The OCV measured with the sequential application of 1-14 (8 μL vol.) droplets having different D₂O concentrations is shown in FIG. 14. The response on application of a subsequent 8 μL vol. pure H₂O droplet is also shown. These results indicate that the apparatus or device can readily detect trace amounts of D₂O at concentrations as low as ˜100 ppm.

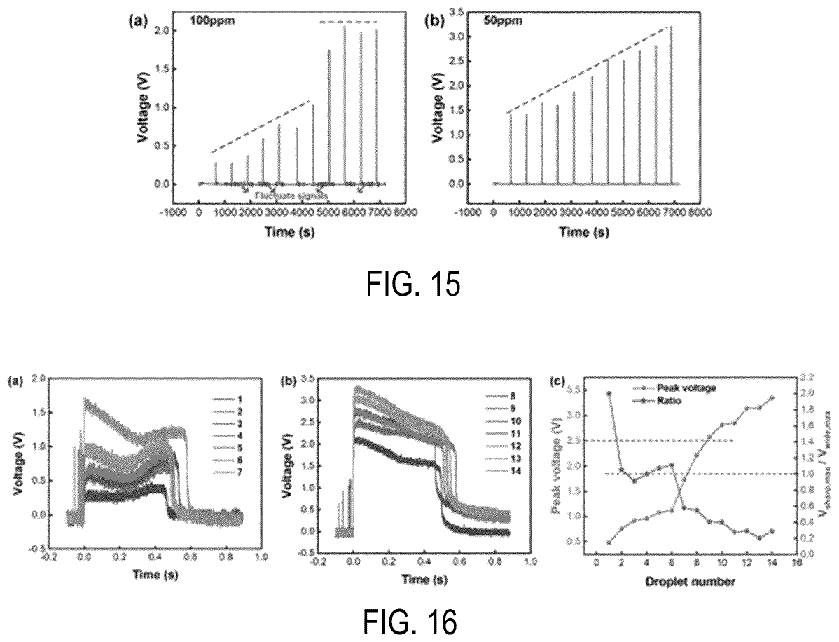

FIG. 15 shows details of the OCV response of the detector when it is exposed to D₂O—H₂O mixtures containing 100 ppm and 50 ppm of D₂O. The OCV generated from droplets of the 100 ppm D₂O mixture increases quasi-linearly from ˜0.25 V to ˜1.0 V as the first seven droplets are applied. The OCV then jumps to ˜1.8 V for eight or more droplets. With droplets containing 50 ppm of D₂O, the OCV increased linearly from 1.4 V to 3.1 V as a series of droplets were applied sequentially. This response is similar to that observed during the application of pure H₂O droplets as shown in FIG. 5(a) as described above. The OCV response generated from application of droplets of a 100 ppm D₂O—H₂O mixture, measured using the oscilloscope with a bandwidth of 200 KHz, is shown in FIG. 16. The amplitude of V_{wide} gradually increases with application of the first few droplets and then increases significantly on application of several additional droplets. The ratio Vₛₕₐᵣₚ/V_{wide}, is >1 for the first several pulses and then exceeds unity for following pulses. Overall, the OCV response to the concentration of D₂O in D₂O—H₂O mixtures indicates that a reaction with D₂O, DH and D⁺ replaces the active surface sites on Al₂O₃ that facilitate the generation of the streaming current. This reaction occurs even for one 8 μL droplet when low concentrations (e.g., 100 ppm of D₂O) are present in the mixture. The application of subsequent droplets reduces this effect, returning the OCV toward that generated from application of pure H₂O droplets.

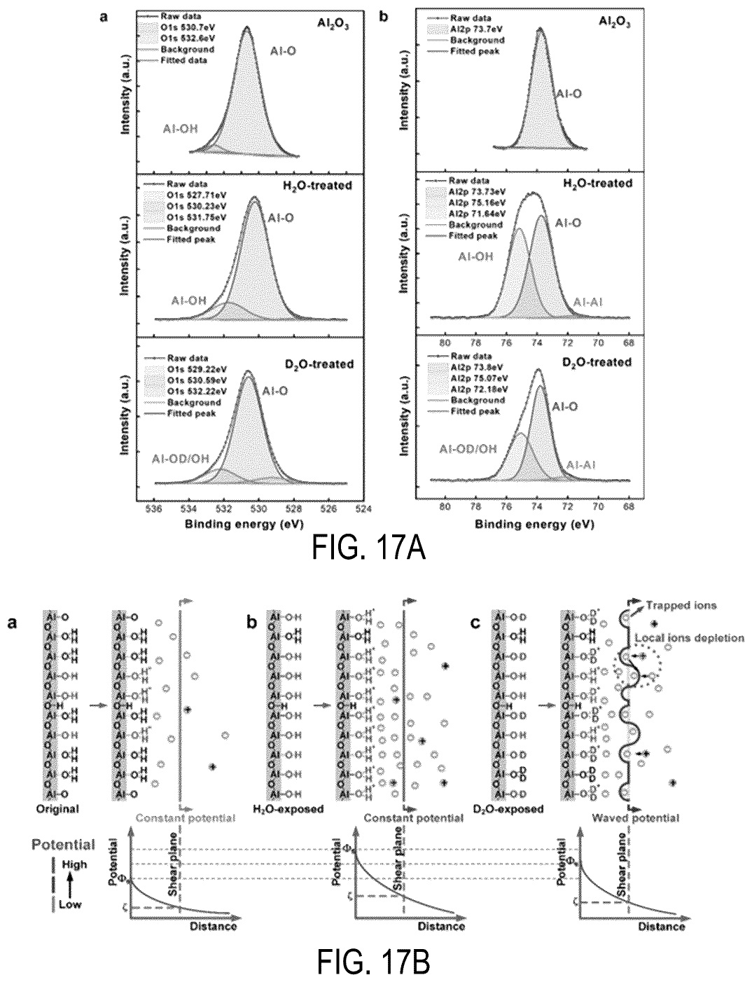

V_{wide} originates from a streaming current which appears from the interaction of water molecules with Al₂O₃ nanoparticles and the flow of water through the nanochannels in the porous layer. The nano porous Al₂O₃ film before and after exposure to 100 uL of H₂O and D₂O were analyzed using XPS. It shows that the concentration of Al—OH/Al—OD bonds was increased when the Al₂O₃ was exposed to H₂O and D₂O when compared to the original Al₂O₃, indicating that some hydroxyl groups are produced during the interaction of Al₂O₃ nanoparticles and H₂O/D₂O. The ratios of Al—OH/OD and Al—O are 0.83 and 0.63 for the H₂O-processed and D₂O-processed Al₂O₃ nanoparticles, respectively, which indicated that high-proportioned hydroxyl groups of Al—OH appear after the H₂O-exposure treatment than that (Al—OD) produced from D₂O-exposured alumina. This lays the foundation of the sensing mechanism to distinguish between D₂O and H₂O. It is evident that the introduction of isotopologues of water into the porous Al₂O₃ layer can influence the distribution of surface functional groups producing local inhomogeneities and variations in surface charge. As seen in FIG. 17A and FIG. 17B, the original porous Al₂O₃ film contains a low concentration of Al—OH groups and exhibits a lower value of the zeta potential, ζ. ζ increases as the concentration of Al—OH groups rises when the first few water droplets are introduced into the system. Maximum surface coverage with Al—OH groups is achieved in the equilibrium state increasing the surface potential resulting in high OCV. The Al—OH surface groups obtained from a droplet of H₂O will bond in a similar chemical environment to that of pre-existing Al—OH groups obtained from atmospheric humidity. This ensures that the zeta potential is spatially uniform along the surface of the nanochannel.

When the —OH in these surface groups is substituted with D atoms, or another isotopic species such as ¹⁸O the surface composition becomes inhomogeneous, introducing localized regions where the potential varies from the average value for a surface containing only —OH groups. In this case, these variations in the local potential act as “traps” that can attract negatively or positively charged ions depending on the local electric field gradient. For a flowing liquid containing charges, the existence of traps will act to reduce the streaming current, decreasing the amplitude of V_{wide}. This effect will occur when D₂O is introduced into the system, resulting in the low OCVs observed compared to those obtained in —OH dominated Al₂O₃surfaces.

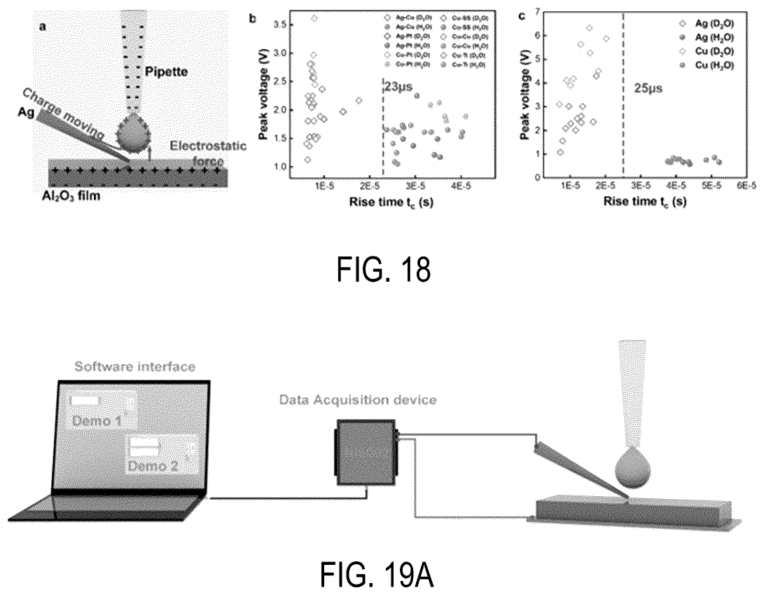

Vₛₕₐᵣₚ arises from charges contained in the droplet and collected by the electrodes (as shown in FIG. 18), and is found effective to detect D₂O. The mechanism to distinguish D₂O from H₂O not only works in the Ag—Al₂O₃-carbon system, but also effective with a metal-metal system and even one electrode system (FIG. 18). It shows the clear difference in the rise time of Vₛₕₐᵣₚ between the H₂O and D₂O results.

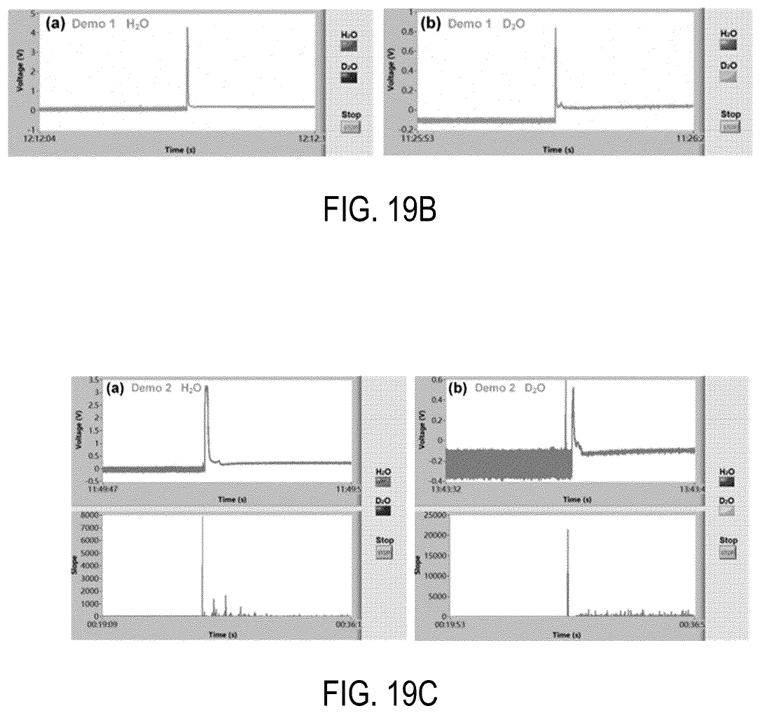

A practice D₂O sensing system (FIG. 19A) was also developed. The system includes three parts: software programmed using LabVIEW, a data acquisition card of NI USB-6002 (maximum sampling rate of 50 kHz), and the sensor developed in this work. Two diagnostics, the amplitude of V_{wide}(Demo 1) and time-dependent characteristics of Vₛₕₐᵣₚ (Demo 2) are selected for the demonstration. The application of water droplets to the sensor was kept with 8 μL volume and 10 min interval, and separate sensors were used for H₂O and D₂O, respectively. With a sampling rate of 200 Hz, V_{wide} signals were acquired as shown in FIG. 19B, which can show the presence of H₂O and D₂O. When the sampling rate was set at 50 kHz, the signal corresponding to Vₛₕₐᵣₚ and dVₛₕₐᵣₚ/dt can be acquired. As shown in FIG. 19C, the presence of H₂O and D₂O produce different and distinct signals. These demonstrations confirm that the heavy water sensor developed in this work can be used to distinguish between H₂O and D₂O.

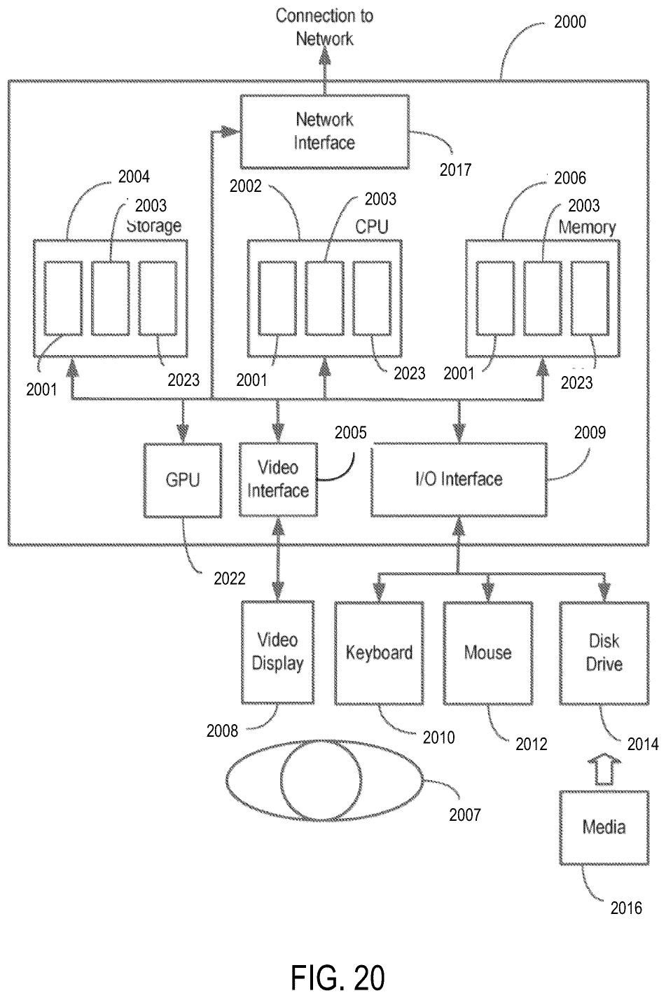

Now referring to FIG. 20, shown is a schematic block diagram of a generic computing device that may provide a suitable operating environment in one or more embodiments. A suitably configured computer device, and associated communications networks, devices, software and firmware may provide a platform for enabling one or more embodiments as described above. By way of example, FIG. 20 shows a generic computer device 2000 that may include a central processing unit (“CPU”) 2002 connected to a storage unit 2004 and to a random access memory 2006. The CPU 2002 may process an operating system 2001, application program 2003, and data 2023. The operating system 2001, application program 2003, and data 2023 may be stored in storage unit 2004 and loaded into memory 2006, as may be required. Computer device 2000 may further include a graphics processing unit (GPU) 2022 which is operatively connected to CPU 2002 and to memory 2006 to offload intensive image processing calculations from CPU 2002 and run these calculations in parallel with CPU 2002. An operator 2010 may interact with the computer device 2000 using a video display 2008 connected by a video interface 2005, and various input/output devices such as a keyboard 2010, pointer 2012, and storage 2014 connected by an I/O interface 2009. In known manner, the pointer 2012 may be configured to control movement of a cursor or pointer icon in the video display 2008, and to operate various graphical user interface (GUI) controls appearing in the video display 2008. The computer device 2000 may form part of a network via a network interface 2011, allowing the computer device 2000 to communicate with other suitably configured data processing systems or circuits. A non-transitory medium 2016 may be used to store executable code embodying one or more embodiments of the present method on the generic computing device 2000.

In an embodiment, computing device 2000 is operatively connected to the apparatus or device for distinguishing between isotopically substituted liquids, such as H₂O and D₂O, and upon detection of a particular isotopic composition or isotopologue, triggering an alarm as may be required to alert an operator that the particular isotopic composition or isotopologue has been detected.

The present disclosure has provided a description of the fabrication processes, working mechanism, and operating characteristics of a new kind of sensor for heavy water and other isotopologues of water. The sensing process can be triggered and controlled by applying droplets to a hydrophilic and porous middle layer in contact with two electrodes. Electricity is generated in response to the interactions between water molecules and the nanoparticles in the middle layer, and the characteristics of the output voltage signals are sensitive to the isotopic composition of the liquid. In one embodiment, the output voltage of the detector consists of two voltage pulses, Vₛₕₐᵣₚ and V_{wide}, separated in time. Four diagnostic protocols for the presence of D₂O are developed. These are based on 1) the amplitude of the second voltage pulse (V_{wide}) relative to a standard value. 2) the ratio of the amplitudes of these two voltage pulses, Vₛₕₐᵣₚ/V_{wide}. 3) the temporal characteristics of Vₛₕₐᵣₚ. 4) comparison of the FFT spectra of V_{wide} obtained with droplets of D₂O and H₂O. This apparatus or device also shows a high sensitivity to the presence of isotopically substituted water including H₂¹⁸O as the voltage generated is found to be suppressed when sensing D₂O and H₂¹⁸O. Consequently, this disclosure encompasses not only the embodiments explicitly disclosed herein, but also any equivalents that may reasonably suggest themselves to those skilled in the pertinent arts. Thus, this disclosure encompasses all modifications and alternate constructions coming within the scope of this disclosure.

Thus, in an aspect, there is provided an apparatus or device for sensing different isotopes or isotopologues of a liquid. In an embodiment, the apparatus or device is configured to detect the presence of different isotopes or isotopologues of water comprising: i) an inert bottom electrode that may be of flaky or smooth structure, ii) a hydrophilic middle layer incorporating nano- and/or micro-scale porous structures and iii) an inert flaky or cylindrical top electrode.

In another embodiment, the apparatus or device is configured to sense D₂O or other isotopologues of water based on the electrical signals generated during the application of water droplets on the porous nanostructure. Four diagnoses are developed to discriminatorily identify H₂O and D₂O from the characteristics of the amplitude of voltage output, relationship between two sequential voltage signals, temporal characteristics of voltage signal and the frequency components present in the voltage pulses.

In another embodiment, the middle layer is hydrophilic and porous. The middle layer is fabricated by nano- and/or micro-materials and contains a high density of nano- and/or micro-channels that can allow water to flow through.

In another embodiment, the apparatus or device is configured such that droplets applied on the middle layer will trigger a reaction between the top electrode, nanomaterial, and water molecules, to generate two sequential voltage pulses. The first voltage pulse originates from the interaction between top electrode, surface of the nanostructure and the water droplets, and the second voltage pulse originates from the interaction between the nanomaterial and the flow of water containing positive and negative ions.

In another embodiment, the hydrophilic layer is configured to easily absorb a liquid, such as water, on the surface of the material, and can easily flow through the matrix or compacted structure with nano- and/or micro-scale porosity dragging the ions from the upstream and generating a streaming current between two electrodes.

In another embodiment, the relatively or substantially inert electrodes include carbon, gold, platinum, silver, copper, titanium or stainless steel which presents no or very small reaction with water at room temperature in an air ambient at normal atmosphere pressure. The electrodes are used for electrons transfer and the electricity is mainly generated because of the middle layer.

In another embodiment, the property and shape of the top electrode is optimized for liquid such as water flowing into the nanomaterial layer. The top electrode needs to be super-hydrophilic and porous for fast water penetration for a flaky material and may include a tip electrode. Depend on the specific electrode material, the electrode may be one of the action parts for the generation of first voltage pulse.

In another embodiment, the porous middle layer comprises a matrix or a compacted structure of nanomaterial that contains nano- and/or micro-channels between individual nano- and micro-scale structures rendering the middle layer porous to water and facilitating the transmission of water from the top electrode to the bottom electrode. The nano-scale and micro-scale here refers to a dimension within the range 1 to 100 nm and 0.1 to 1000 μm, respectively.

In another embodiment, the middle-layer material is compact with abundant nanochannel which can be fabricated by direct compression, vacuum filtration and other method to fabricate a thin nanostructured film.

Thus, in an aspect, there is provided an apparatus for sensing different isotopic compositions or isotopologues of a liquid, comprising: a substantially inert bottom electrode; a hydrophilic middle layer incorporating a porous structure configured to detect the different isotopic compositions or isotopologues of the liquid; and a substantially inert top electrode; wherein, in use, the hydrophilic middle layer produces an electrical output signal of a particular isotopic composition or isotopologue when a sample of the liquid is applied to the hydrophilic middle layer.

In an embodiment, the electrical output signal comprises voltage signals having an amplitude and time-dependence characteristic of the particular isotopic composition or isotopologue.

In another embodiment, the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

In another embodiment, Vₛₕₐᵣₚ originates from charges produced during a liquid pipetting process being collected by the top electrode, and V_{wide} originates from the generation of streaming current/potential as a liquid flows through the porous hydrophilic middle layer.

In another embodiment, the hydrophilic middle layer comprises a structure assembled from one or more of nano-particles, micro-particles, and wires.

In another embodiment, the hydrophilic middle layer further comprises nano-channels or micro-channels adapted to encourage liquids to rapidly flow from a top side of the hydrophilic middle layer to a bottom side of the hydrophilic middle layer during which a strong streaming current/potential can be generated.

In another embodiment, the thickness of the porous middle layer is controlled by selecting a mass of a sample before compression into the hydrophilic middle layer.

In another embodiment, the hydrophilic middle layer comprises one or more of Al₂O₃ nanoparticles, Al₂O₃ nanowires, TiO₂ nanoparticles/nanowires, ZnO nanoparticles/nanowires, and SiO₂ nanoparticles.

In another embodiment, the hydrophilic middle layer is fabricated from Al₂O₃ nanoparticles having a diameter of 200 nm.

In another embodiment at least one of the electrodes is made from one or more of carbon paper, carbon cloth, gold or gold-coated silicon wafer, platinum or platinum-coated silicon wafer, silver, copper, and stainless steel.

In another embodiment, the bottom electrode is a flaky material with a surface area the same or larger than that of the hydrophilic middle layer.

In another embodiment, the top electrode geometry is configured to permit a rod-shaped or point contact with the hydrophilic middle layer, thereby enabling the application of liquid directly onto the top surface of the hydrophilic middle layer.

In another aspect, there is provided a method of sensing different isotopic compositions or isotopologues of a liquid, comprising: providing a substantially inert bottom electrode; providing a hydrophilic middle layer incorporating a porous structure configured to detect the different isotopic compositions or isotopologues of the liquid; providing a substantially inert top electrode; and measuring an electrical output signal produced by the hydrophilic middle layer when a particular isotopic composition or isotopologue when a sample of the liquid is applied to the hydrophilic middle layer.

In an embodiment, the method further comprises measuring an electrical output signal comprising voltage signals having an amplitude and time-dependence characteristic of the particular isotopic composition or isotopologue.

In another embodiment, the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

In another embodiment, Vₛₕₐᵣₚ originates from charges produced during a liquid pipetting process being collected by the top electrode, and V_{wide} originates from the generation of streaming current/potential as a liquid flows through the porous hydrophilic middle layer.

In another embodiment, there is provided a system for sensing different isotopic compositions of isotopologues, the system having a processor, memory, and storage, and comprising: a sensor for sensing different isotopic compositions or isotopologues of a liquid, the sensor having: a substantially inert bottom electrode; a hydrophilic middle layer incorporating a porous structure configured to detect the different isotopic compositions or isotopologues of the liquid; and a substantially inert top electrode; wherein, in use, the hydrophilic middle layer produces an electrical output signal of a particular isotopic composition or isotopologue when a sample of the liquid is applied to the hydrophilic middle layer, and the system detects the characteristic electrical output signal of a particular isotopic composition or isotopologue.

In an embodiment, the electrical output signal comprises voltage signals having an amplitude and time-dependence characteristic of the particular isotopic composition or isotopologue, and upon detection and identification of the particular isotopic composition or isotopologue, triggering an alarm as required.

In another embodiment, the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

In another embodiment, Vₛₕₐᵣₚ originates from charges produced during a liquid pipetting process being collected by the top electrode, and V_{wide} originates from the generation of streaming current/potential as a liquid flows through the porous hydrophilic middle layer.

While various illustrative embodiments of the system, method, and apparatus have been described, it will be appreciated that various modifications and amendments may be made without departing from the scope of the invention.

1. An apparatus for sensing different isotopic compositions or isotopologues of a liquid, comprising:
a substantially inert bottom electrode;
a hydrophilic middle layer incorporating a porous structure configured to detect the different isotopic compositions or isotopologues of the liquid; and
a substantially inert top electrode;
wherein, in use, the hydrophilic middle layer produces an electrical output signal of a particular isotopic composition or isotopologue when a sample of the liquid is applied to the hydrophilic middle layer.

2. The apparatus of claim 1, wherein the electrical output signal comprises voltage signals having an amplitude and time-dependence characteristic of the particular isotopic composition or isotopologue.

3. The apparatus of claim 2, wherein the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

4. The apparatus of claim 3, wherein Vₛₕₐᵣₚ originates from charges produced during a liquid pipetting process being collected by the top electrode, and V_{wide} originates from the generation of streaming current/potential as a liquid flows through the porous hydrophilic middle layer.

5. The apparatus of claim 1, wherein the hydrophilic middle layer comprises a structure assembled from one or more of nano-particles, micro-particles, and wires.

6. The apparatus of claim 5, wherein the hydrophilic middle layer further comprises nano-channels or micro-channels adapted to encourage liquids to rapidly flow from a top side of the hydrophilic middle layer to a bottom side of the hydrophilic middle layer during which a strong streaming current/potential can be generated.

7. The apparatus of claim 6, wherein the thickness of the porous middle layer is controlled by selecting a mass of a sample before compression into the hydrophilic middle layer.

8. The apparatus of claim 1, wherein the hydrophilic middle layer comprises one or more of Al₂O₃ nanoparticles, Al₂O₃ nanowires, TiO₂ nanoparticles/nanowires, ZnO nanoparticles/nanowires, and SiO₂ nanoparticles.

9. The apparatus of claim 8, wherein the hydrophilic middle layer is fabricated from Al₂O₃ nanoparticles having a diameter of 200-300 nm.

10. The apparatus of claim 1, wherein at least one of the electrodes is made from one or more of carbon paper, carbon cloth, gold or gold-coated silicon wafer, platinum or platinum-coated silicon wafer, silver, copper, and stainless steel.

11. The apparatus of claim 1, wherein the bottom electrode is a flaky material with a surface area the same or larger than that of the hydrophilic middle layer.

12. The apparatus of claim 1, wherein the top electrode geometry is configured to permit a rod-shaped or point contact with the hydrophilic middle layer, thereby enabling the application of liquid directly onto the top surface of the hydrophilic middle layer.

13. A method of sensing different isotopic compositions or isotopologues of a liquid, comprising:
providing a substantially inert bottom electrode;
providing a hydrophilic middle layer incorporating a porous structure configured to detect the different isotopic compositions or isotopologues of the liquid;
providing a substantially inert top electrode; and
measuring an electrical output signal produced by the hydrophilic middle layer when a particular isotopic composition or isotopologue when a sample of the liquid is applied to the hydrophilic middle layer.

14. The method of claim 13, further comprising measuring an electrical output signal comprising voltage signals having an amplitude and time-dependence characteristic of the particular isotopic composition or isotopologue.

15. The method of claim 14, wherein the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

16. The method of claim 15, wherein Vₛₕₐᵣₚ originates from charges produced during a liquid pipetting process being collected by the top electrode, and V_{wide} originates from the generation of streaming current/potential as a liquid flows through the porous hydrophilic middle layer.

17. A system for sensing different isotopic compositions of isotopologues, the system having a processor, memory, and storage, and comprising:
a sensor for sensing different isotopic compositions or isotopologues of a liquid, the sensor comprising:
a substantially inert bottom electrode;
a hydrophilic middle layer incorporating a porous structure configured to detect the different isotopic compositions or isotopologues of the liquid; and
a substantially inert top electrode;
wherein, in use, the hydrophilic middle layer produces an electrical output signal of a particular isotopic composition or isotopologue when a sample of the liquid is applied to the hydrophilic middle layer, and the system detects the characteristic electrical output signal of a particular isotopic composition or isotopologue.

18. The system of claim 17, wherein the electrical output signal comprises voltage signals having an amplitude and time-dependence characteristic of the particular isotopic composition or isotopologue, and upon detection and identification of the particular isotopic composition or isotopologue, triggering an alarm as required.

19. The system of claim 18, wherein the electrical output signal comprises two sequential voltage signals comprising a first voltage pulse (Vₛₕₐᵣₚ) having a time duration of hundreds of microseconds, and a second voltage pulse (V_{wide}) having a time duration of hundreds of milliseconds.

20. The system of claim 19, wherein Vₛₕₐᵣₚ originates from charges produced during a liquid pipetting process being collected by the top electrode, and V_{wide} originates from the generation of streaming current/potential as a liquid flows through the porous hydrophilic middle layer.
